(19) United States
(12) Patent Application Publication
(10) Pub. No.: US 2019/0367342 A1
(43) Pub. Date: Dec. 5, 2019

(54) DEVICE FOR TREATING CONTAINER CLOSURES

(71) Applicant: KRONES AG, Neutraubling (DE)

(72) Inventors: Juergen SOELLNER, Neutraubling (DE); Holger MUELLER, Neutraubling (DE)

(21) Appl. No.: 16/431,956

(22) Filed: Jul. 17, 2019

(30) Foreign Application Priority Data
Jun. 5, 2018 (DE) 102018113291.3

(51) Int. Cl. B67B 3/00; A61L 2/20; B67C 7/00; B65G 43/10

(52) U.S. Cl. CPC B67B 3/003; B65G 43/10; B67C 7/0086; A61L 2/208

(57) ABSTRACT

A device for treating container closures, including sterilizing container closures for closing containers in a beverage filling plant. The device may include a treatment chamber for treating container closures, and a transport device for transporting container closures through the treatment chamber. The transport device may include a linear drive.

### TECHNICAL FIELD

The present invention relates to a device for treating container closures, in particular for sterilizing container closures for closing containers in a beverage filling plant.

### STATE OF THE ART

In beverage filling plants it is generally known to treat container closures before they are attached to filled containers in order to close these containers. In particular, the container closures are sterilized so that no germs or other forms of contamination can intrude via the container closures into the filled containers, and in particular into the filling product accommodated in the containers.

In conventional plants it is known to sort the container closures into the desired spatial orientation by means of a cascade conveyor, and at the same time elevate them, then convey the container closures into a treatment chamber in order to sterilize them. The treatment chamber can for example be disposed above a clean room, in order to enable the closures to be conveyed to a capper disposed in the clean room by gravity alone.

In this context it is for example known in aseptic beverage filling plants to utilize container closure disinfection systems, by means of which container closures, which are to be attached to containers that have been filled, are sterilized and/or disinfected. In such treatment chambers, it is usual to use peracetic acid and/or hydrogen peroxide in vapor form to sterilize the container closures. These container closure sterilization systems are for example provided with their own guide chute, along which the container closures that are to be sterilized and/or disinfected slide due to gravity and are impinged while sliding with the sterilizing agent and/or disinfection agent.

This type of device for treating container closures has a considerable height, and requires a hall with a ceiling height of up to 10 meters in order to accommodate it. In order to achieve the necessary throughput of container closures, a plurality of container closures are simultaneously present in the guide chute in the treatment chamber. Consequently, the pressure from the other container closures that are conveyed through the guide chute can lead to container closures wedging or jamming in the guide chute.

The guide chute needs to be suitably designed for the applicable type of container closure in order to achieve problem-free transport of the container closures, in the correct orientation, through the treatment chamber and to the capper. When there is a change in the type of container that is used, the guide chute must therefore be exchanged. Alternatively, differing guide chutes are provided, parallel to each other, in the treatment chamber, and it is possible to switch between these guide chutes.

At the end of the treatment chamber in which the treatment of the container closures takes place, the guide chute can have a stopper which prevents a container closure from sliding further through the guide chute. This is necessary in order to ensure that each container closure—including the first of these, which is not on top of a column of container closures—has spent the necessary treatment time in the treatment chamber.

Due to the aggressive environment within the treatment chamber, which is in particular due to the prevailing temperature inside the chamber and the concentration of the sterilizing agent, the dwell time of the container closures in the treatment chamber must be adhered to with an appropriate degree of precision.

If the dwell time in the treatment chamber is too short, there is a risk that the container closures will not be properly sterilized. On the other hand, container closures that spend too long in the treatment chamber, for example due to the container closures jamming in the guide chute or due to a stoppage in the plant, must be rejected, and cannot be used to close a container. This is because, for example, the material of the container closures has been thermally treated for too long, or has been damaged by excessive exposure to the treatment agent. If the container closures spend too long in the treatment chamber, they can also deform in the guide chute due to the temperature and the pressure from the other container closures.

Because the guide chute can convey container closures by gravity only in one direction, those which are no longer usable, because for example they have been subjected to overtreatment, must be ejected in a downwards direction. They then normally land on the floor of the isolator of the beverage filling plant. Opening the isolator and removing these container closures is, however, a breach of the clean room status of the isolator, and an interim sterilization is thus required. The attempt is therefore made to avoid this.

### SUMMARY OF THE INVENTION

Proceeding from the known state of the art, an object of the present invention is to provide an improved device for treating container closures, in particular for sterilizing container closures for containers in a beverage filling plant.

This object is achieved by a device for treating container closures, preferably for sterilizing container closures for containers in a beverage filling plant, with the features of claim 1. Advantageous further developments arise from the dependent claims, the description and the attached figures.

Accordingly, a device for treating container closures, preferably for sterilizing container closures for closing containers in a beverage filling plant, is proposed, comprising a treatment chamber for treating container closures, and a transport device for transporting container closures through the treatment chamber. According to the invention, the transport device comprises a linear drive, wherein the linear drive can be operated in a direction of conveyance and in a direction counter to the direction of conveyance.

Due to the fact that the transport device comprises a linear drive, the movement of a single container closure or a plurality of container closures through the treatment chamber can be individually and variably adjusted. Consequently, it is possible to vary the treatment time of the container closures in the treatment chamber, and/or adapt this to the applicable type of container closure. By this means a single size of treatment chamber can be used for a variety of container closure types. Furthermore, it is unnecessary to use differing guide chutes, which provide the various types of container closures with differing transit paths along with differing treatment times, in a single treatment chamber.

Furthermore, due to the fact that the transport device comprises a linear drive, it is no longer necessary for the container closures that are to be treated first to be lifted via a mass flow conveyor and then allowed to slide downwards by gravity through a guide chute. Instead, the linear drive also permits horizontal movements and vertical movements both in and counter to the direction of gravity. The treatment of the container closures can therefore also take place close to the ground. It is consequently unnecessary to provide space or technical measures in the elevated area for transportation, sorting and return by gravity, or for inspecting and servicing the plant components and devices that are necessary for this purpose.

Due to the fact that the linear drive can be operated both in a direction of conveyance and in a direction counter to the direction of conveyance, it is also possible for container closures that are present in the treatment chamber to be moved back out of the treatment chamber, in a direction counter to the direction of conveyance. In particular, in the event of a stoppage in the plant, container closures that are present in the treatment chamber can be moved back out of the chamber, and subsequently, when the plant resumes operation, can be again conveyed into the treatment chamber, so that the interrupted treatment of the container closures can be resumed or restarted from the beginning.

Due to the fact that the container closures are conveyed out of the treatment chamber in a backwards direction, there is no need to provide additional transport sections or buffer sections downstream of the treatment chamber. Consequently, the capper for closing containers with the container closures can be disposed immediately downstream of the treatment chamber. By this means a particularly compact design of a plant comprising the device is achieved.

In the event of a stoppage of a plant that comprises the device for treating container closures, for example a beverage filling plant, container closures that are present in the treatment chamber can thus be actively moved out of the treatment chamber by the linear drive, so that overtreatment or damage to the container closures can be avoided. Preferably, the container closures that are moved out of the treatment chamber during the plant stoppage can be conveyed back into the treatment chamber for treatment after the plant has restarted. In consequence, the number of containers that are rejected in the event of a plant stoppage can be significantly reduced.

Due to the active transport of the container closures by means of the linear drive, deformation of the container closures can also be avoided, since there is no pressure on the individual container closures from other container closures, which instead can be conveyed substantially without touching each other.

In addition, by means of the linear drive a downstream capper can be supplied with container closures at exactly the correct intervals. It is thus unnecessary to provide an additional stopper between the transport device and the capper or a Pick&Place station or positioning system for the capper.

The introduction and discharge of container closures into the treatment chamber and out of the treatment chamber via airlocks can be achieved in a particularly efficient manner by means of the linear drive. In particular if the treatment chamber has a multi-door airlock for introducing and/or discharging container closures, a container closure that is introduced through a first door of the airlock can be halted or decelerated in the airlock until the first door is again closed and the second door has then been opened. A similar procedure can be also used when a container closure is discharged from the treatment chamber. In addition, the consumption of disinfecting agent can be reduced due to the improved sealing of the airlock, since losses at the airlock are reduced.

Accordingly, by means of the equipping of the transport device with a linear drive, a device for treating a container closure with a particularly compact, efficient and simple construction can be provided.

According to a further preferred embodiment, the transport device has a long stator and at least one carriage, which can be individually driven via the long stator, for accommodating at least one container closure that is to be treated, wherein preferably a plurality of carriages are provided. It is further possible that a carriage is designed to accommodate a plurality of container closures.

The linear drive can be designed in the form of a linear motor, wherein a stator is provided in the form of a long stator, by means of which the transport path is defined, and the carriages are individually moved on this long stator.

By means of the carriage, which can be individually driven, it is thus possible individually to control and/or adjust the position and the movement of a single container closure or a plurality of container closures into and through the treatment chamber. It is also possible by this means for a container closure to be taken from a supply device with exactly the correct timing, and for a container closure to be transferred to the capper with exactly the correct timing. The long stator preferably provides a movement path for the carriage, i.e. a circuit or a main transport line for the carriage.

It is also possible for more than one container closure to be transported with each carriage. In this manner efficient transportation of the container closures can be achieved with a reduced number of carriages.

According to a further preferred embodiment, the long stator can also be disposed outside a wall of the treatment chamber, and the carriage can be guided in the treatment chamber via a guide on the other side of the wall. By this means the long stator is prevented from coming into contact with the treatment medium in the treatment chamber.

If, in accordance with a further preferred embodiment, a carriage has a carrier for taking a container closure from a supply chute, the carriage can further function as a stopper on the supply chute.

Preferably, when a container closure is taken from the supply chute, for example a supply chute of a sorter, a carriage is always positioned at the outlet of the supply chute, in order to prevent a container closure from falling out of the supply chute. The carrier serves to enable a container closure which is transferred from the supply chute to a carriage that has a carrier to be reliably carried by the carriage when the carriage moves away from the supply chute. In addition, the carrier of the next carriage, which is moved into the position of the previous carriage after that carriage has moved away from the supply chute, can take over a further container closure, and preferably, by means of its carrier, prevent additional container closures from sliding out or discharging from the supply chute.

Additionally, if the transport device has a buffer for the interim storage of container closures, it is possible temporarily to store container closures which, for example, are conveyed out of the treatment chamber during a stoppage of the plant. By this means it is also possible, in the event of faults in components of the plant or the device, for example in the event of disruptions in the supply of container closures to the transport device, to continue operation of the plant without a significant interruption.

According to a further preferred embodiment, the transport device has a track switch, wherein the buffer is connected to a main transport line of the transport device via the track switch, wherein preferably a first track switch for connecting the buffer is provided at a first position in the main transport line, and a second track switch for connecting the buffer is provided at a second position in the main transport line. By this means it is possible for container closures that are guided out of the treatment chamber to be conveyed into the buffer via the track switch, preferably the first track switch, and after interim storage to be conveyed back to the main transport line, again via a track switch, either the first track switch or the second track switch. The second track switch additionally enables the main transport line, which in normal operation forms a circuit, to be used at least in part as an additional buffer section.

If the transport device is configured to supply the container closure directly to a capper for closing a container with a container closure, it is possible to provide a particularly simple design of a plant comprising the device, particularly a beverage filling plant comprising the device.

According to a further preferred embodiment, the treatment chamber is configured as a sterilization chamber for containers, preferably for preforms of the containers, and additionally for the container closures for closing the containers. By this means it is possible to treat both the containers, preferably the preforms of the containers, and the container closures in a common treatment chamber. This is possible firstly because the linear drive enables treatment to take place close to the ground, and secondly because the dwell time or treatment time of the container closures and the containers in the treatment chamber can be adjusted individually and independently of each other, due to the fact that the positioning and/or speed of movement of the container closures that are to be treated in the treatment chamber can be individually controlled.

In other words, a dwell time or treatment time of the container closures is substantially independent of the dwell time or treatment time of the containers or preforms in the same treatment chamber. As a result, it is possible to dispense with a further treatment chamber for the container closures or the containers, which would otherwise need to be provided, and thereby also dispense with a second vaporizer for the sterilization medium, a second air conditioning device with a heat exchanger, and a regulation system for this second treatment chamber, together with a monitoring device for the above-mentioned components. A plant which comprises the proposed device can thus, by comparison with conventional plants, have a more compact and simpler design. In addition, its process reliability is increased. Furthermore, a device that is designed in this way requires a maximum construction height that is lower than that of conventional devices for treating container closures.

In a preferred further embodiment, each carriage of a plurality of carriages is individually controllable. Thus the speed of each carriage along its path can be individually adjusted and controlled. In this manner, for example, in the event of a stoppage in the plant, the carriages holding closures which have already travelled more than half of the treatment section in the treatment chamber can be driven in a reverse direction at a higher speed, in order to avoid exposing the closures to an excessively long treatment period as they travel in reverse. When the plant resumes operation, the closures that have been driven in reverse are again driven through the treatment chamber under the ideal conditions, in particular for the remaining time that is still necessary or intended.

On a return path between the capper and a closure supply for supplying the carriages with the new caps that are to be treated, the carriages are preferably driven at a high to very high speed, in particular by comparison with their speed between the closure supply and the treatment chamber, and within the treatment chamber. They thus complete this section of the path rapidly, and by this means fewer carriages can be used on the line as a whole, while the treatment output remains the same.

In order to clean the container closures before they are conveyed into the treatment chamber, in particular to free them from coarse dirt, a cleaning device can be disposed upstream of the treatment chamber for removing dirt from a container closure that is to be treated. The cleaning device can preferably be designed as a rinsing device or blowing device, which uses a fluid rinsing medium or a gas, preferably clean air, to clean or pre-clean the container closures.

In particular, pre-cleaning of container closures before they are conveyed into the treatment chamber can be still more effective if the transport device has, relative to its main direction of travel, a twisted section, which is designed such that a container closure that is transported by the transport device undergoes a change in its orientation in the twisted section, wherein the twisted section is preferably disposed in the region of the cleaning device. Preferably, in the twisted section a container closure is at least in part held in or on the carriage.

In this case, the container closures are preferably oriented on the transport device in a horizontal position upstream of, i.e. in advance of, the twisted section, and undergo in the twisted section a change in their orientation, and preferably a tipping or pivoting displacement, so that they are disposed alongside or below the transport device. Preferably, in this case the cleaning device is also disposed below the transport device, so that the cleaning medium from the cleaning device, preferably the cleaning fluid, gas or gas mixture, can be sprayed or blown from below onto the container closure that is to be cleaned. This further enables coarse dirt particles to separate from the container closure, either before cleaning starts by the action of gravity, or at the latest following the cleaning in the direction of gravity.

### BRIEF DESCRIPTION OF THE FIGURES

Preferred further embodiments of the invention are more fully explained by the description below of the figures. The figures show:

FIG. 1 a schematic representation of a device for treating container closures;

FIG. 2 a schematic representation of the device from FIG. 1 in normal operation;

FIG. 3 a schematic representation of the device from FIG. 1 shortly after a stoppage of the beverage filling plant;

FIG. 4 a schematic representation of a device for treating container closures according to a further embodiment; and

FIG. 5 a schematic representation of a device for treating container closures according to another further embodiment.

### DETAILED DESCRIPTION OF EXAMPLES OF PREFERRED EMBODIMENTS

Examples of preferred embodiments are described below with the aid of the figures. In the figures, elements which are identical or similar, or have identical effects, are designated with identical reference signs. In order to avoid redundancy, repeated description of these elements is in part dispensed with.

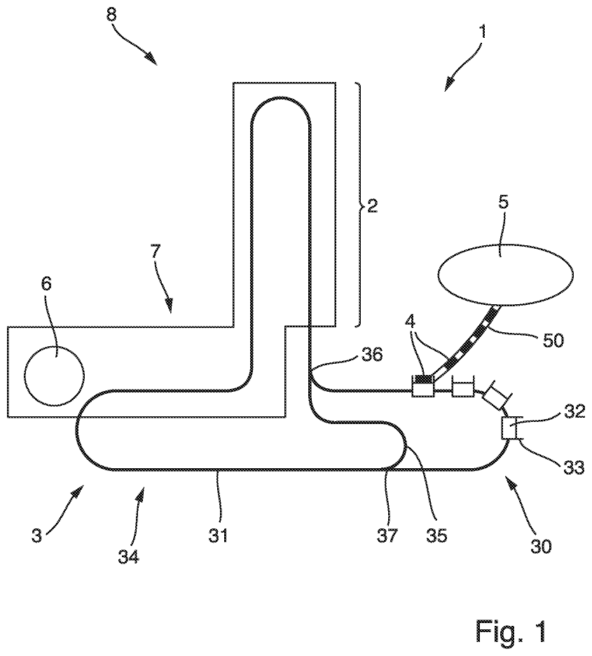

FIG. 1 shows schematically a device 1 for treating container closures 4. The device 1 is integrated in a beverage filling plant 8. The beverage filling plant 8 also has a sorting device 5 for sorting container closures 4, as well as an isolator 7 and a capper 6, disposed in the isolator 7, for closing containers with the container closures 4.

The sorting device 5, the capper 6 and the isolator 7 can also be designed as parts of the device 1.

The device 1 has a treatment chamber 2, which in this case is configured to sterilize the container closures 4. In this case the treatment chamber 2 is combined with the isolator 7 of the beverage filling plant 8. Alternatively, the treatment chamber 2 can also be provided separately from the isolator 7. The device 1 also has a transport device 3, which comprises a linear drive 30. The linear drive 30 has a long stator 31 and a plurality of carriages 32, each of which can be driven individually via the long stator 31, for accommodating container closures 4 that are to be treated. The linear drive 30 is accordingly constructed in the form of a linear motor, with a long stator 31 and a plurality of carriages 32 which move on it and can be individually driven.

Each carriage 32 has a carrier 33 for taking a container closure 4 that is to be transported from a supply chute 50 of the sorting device 5. The carriage 32 and the carrier 33 can also be designed such that in each case more than one container closure 4 is taken and transported by the carriage 32. For example two or more, for example 4, 6 or 8, container closures 4 can be transported on the carriage 32.

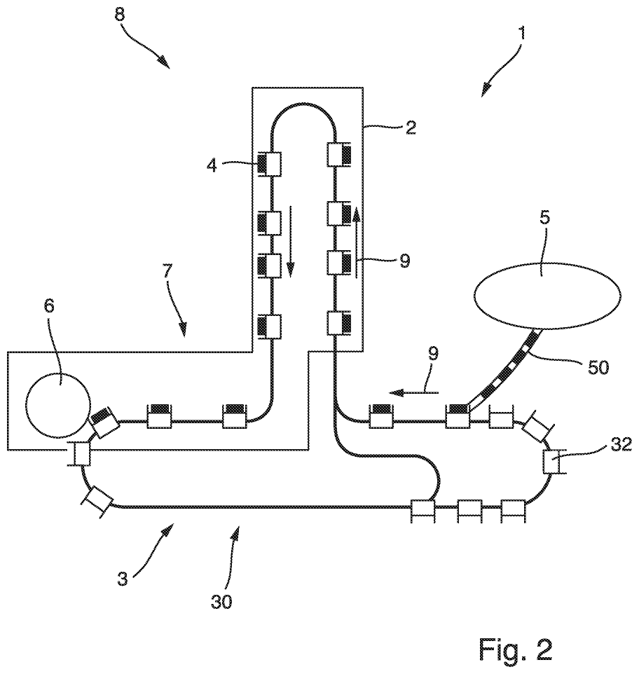

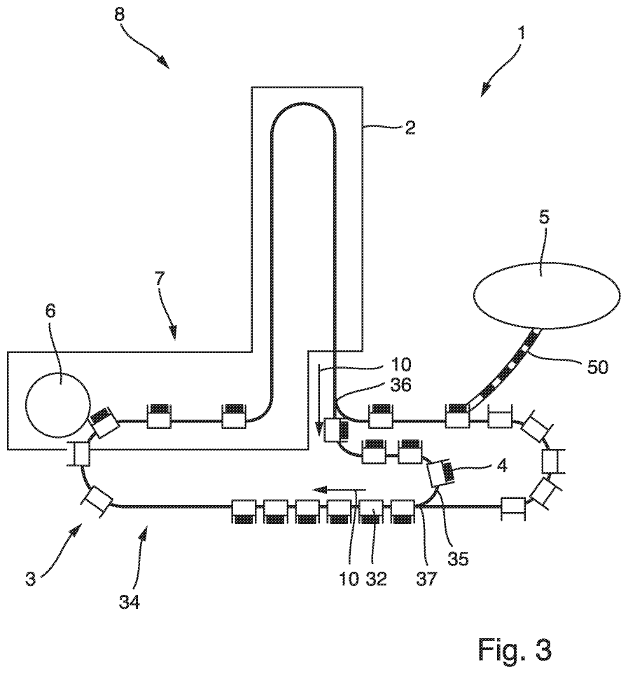

In this embodiment the linear drive 30 is designed such that it can be operated both in a direction of conveyance (see FIG. 2) and in a direction counter to the direction of conveyance (see FIG. 3). In other words, the carriages 32 can be displaced along the transport device 3 in two directions.

The transport device 3 further has a buffer 35, which is provided for the interim storage of container closures 4. The buffer 35 is connected to a main transport line 34 of the transport device 3 via a first track switch 36 and a second track switch 37. The first track switch 36 is disposed on the main transport line 34 between the supply chute 50 and the treatment chamber 2; the second track switch 37 is disposed on the transport device 3 downstream of the capper 6 and the isolator 7.

FIG. 2 shows schematically the device 1 from FIG. 1 in normal operation, in which the transport device 3 moves the carriages 32 individually in the direction of conveyance 9. Container closures 4 that are supplied from the sorting device 5 via the supply chute 50 to the transport device 3 are individually taken at the end of the supply chute 50 by in each case one carriage 32. After a container closure 4 has been taken by a carriage 32 at the end of the supply chute 50, the carriage 32 that carries the container closure 4 is passed through an airlock into the treatment chamber 2. In the treatment chamber 2, the container closure 4 is impinged with a vaporous treatment medium, in this case vaporized hydrogen peroxide. Alternatively, another known treatment medium, for example peracetic acid, can also be used in the treatment chamber 2 to achieve the treatment of the container closures.

The dwell time or treatment time of each container closure 4 in the treatment chamber 2 is governed by the speed that is set for the individual carriage 32, and can therefore be adjusted precisely. Overtreatment or insufficient treatment can thereby be effectively avoided. After the treated container closures 4 leave the treatment chamber 2 through an airlock they are conveyed further in the isolator 7 and transferred directly and at exactly the required intervals to the capper 6. After its container closure 4 has been transferred to the capper 6, the carriage 32 leaves the isolator 7 and is moved along the transport device 3, following the circuit of the main transport line 34 further in the direction of, and up to, the supply chute 50, in order to enable it to pick up a new container closure 4 that is to be treated. On this “return trip” each empty carriage 32 can be moved at a high speed, in order in this manner to minimize the number of carriages 32 that are required.

FIG. 3 shows schematically the device 1 from FIG. 1 shortly after a stoppage of the beverage filling plant 8. In order to avoid overtreatment of container closures 4 that were already present in the treatment chamber 2 when the stoppage occurred, they are moved back out of the treatment chamber 2 in the direction 10 counter to the normal direction of conveyance 9 (see FIG. 2). In this case those carriages 32 that carry closures which have already undergone more than 50% of their treatment are conveyed out of the treatment chamber 2 at a particularly high speed.

The carriages 32 reach and enter the buffer 35 via the first track switch 36. Because more carriages 32 were present in the treatment chamber 2 than can be accommodated in the buffer 35, some of the carriages 32 that were present in the treatment chamber 2 when the plant stoppage occurred have been conveyed further via the second track switch 37 onto the main transport line 34. In other words, the region of the main transport line 34 that is provided for the return conveyance of the carriages 32, after they have left the isolator 7, serves as an additional buffer, i.e. as an extension of the buffer 35.

When the stoppage in the plant has ended, the carriages 32 that are present in the buffer 35 and in the additional buffer are again conveyed in the direction of conveyance 9 up to and into the treatment chamber 2, so that the container closures 4 can undergo correct sterilization. When the buffer 35 and the additional buffer have been emptied, new container closures 4 are again taken from the supply chute 50 and, as shown in FIG. 2, i.e. in normal operation, moved through the treatment chamber 2.

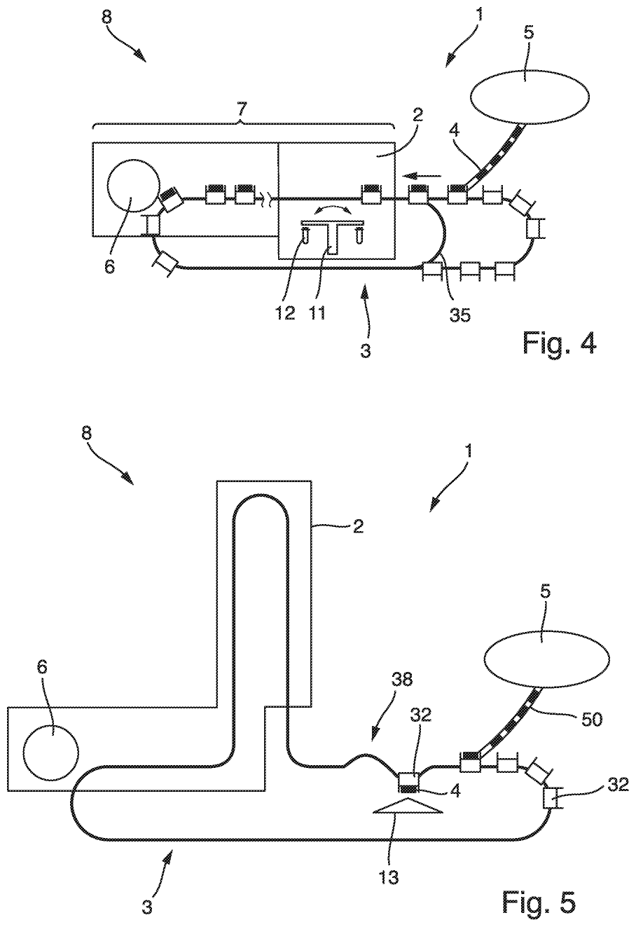

FIG. 4 shows schematically a device 1 for treating container closures 4 according to a further embodiment. It substantially corresponds to the device shown in FIG. 1. The treatment chamber 2, however, is not provided only for treating the container closures 4, but in this embodiment it is configured as a sterilization chamber for container preforms 12 which, after being blow molded and filled with a filling product, are to be closed with the container closures 4 in the capper 6. The treatment chamber 2 is further configured to treat the container closures 4 for closing the containers. Thus sterilization takes place of both the container closures 4, which are moved by means of the transport device 3 through the treatment chamber 2, and the preforms 12, which are also transported through the treatment chamber 2 by means of a preform transport device 11 that is provided separately from the transport device 2. In this case the treatment times of the container closures 4 and the preforms 12 differ, and are adapted to the applicable molded parts. It is possible by this means for both the preforms 12 and the container closures 4 to undergo a treatment with an optimum treatment time in the treatment chamber 2.

FIG. 5 shows schematically a device 1 for treating container closures 4 according to a further embodiment. It substantially corresponds to the device shown in FIG. 1, but in this embodiment no buffer is provided. Alternatively, however, the device 1 shown in FIG. 5 can also comprise a buffer in accordance with the previously described embodiments. Between the supply chute 50 and the treatment chamber 2, the transport device has a twisted section 38. In this twisted section 38, each of the carriages 32 undergoes a change in orientation. While at the end of the supply chute 50 the carriages 32, each with its receptacle for the container closure 4, point upwards, i.e. counter to the direction of gravity, in the twisted section 38 the orientation of the carriages 32 is pivoted such that the carriages 32, with their receptacles, point downwards, i.e. in the direction of gravity. Consequently, a container closure 4 that is held or accommodated in the inverted carriage 32 also points downwards. Below the twisted section 38 a cleaning device 13 is disposed, which removes coarse dirt and dust from the container closures 4 before they enter the treatment chamber 2, in this case by blowing the dirt and dust away with compressed air. After the container closure 4 has been pre-cleaned by the cleaning device 13, the carriage 32 that contains this container closure 4 is further pivoted in the remaining portion of the twisted section 38, so that it again adopts its original orientation.

To the extent applicable, all individual features that are described in the example embodiments can be combined with each other and/or exchanged, without departing from the field of the invention.

### LIST OF REFERENCE SKINS

1 device
2 treatment chamber
3 transport device
30 linear drive
31 long stator
32 carriage
33 carrier
34 main transport line
35 buffer
36 first track switch
37 second track switch
38 twisted section
4 container closure
5 sorting device
5 supply chute
50 capper
6 isolator
7 beverage filling plant
9 direction of conveyance
10 direction counter to the direction of conveyance
11 preform transport device
12 preform
13 cleaning device

1.-10. (canceled)

11. A device for treating container closures, including sterilizing container closures for closing containers in a beverage filling plant, the device comprising:
a treatment chamber for treating a container closure; and
a transport device for transporting the container closure through the treatment chamber, the transport device comprising a linear drive operable in a direction of conveyance and in a direction counter to the direction of conveyance.

12. The device according to claim 11, wherein the transport device includes:
a long stator and
a carriage individually driven via the long stator for accommodating the container closure that is to be treated.

13. The device accordingly to claim 12, wherein the carriage is a first carriage of a plurality of carriages driven via the long stator.

14. The device according to claim 12, wherein the carriage has a carrier for taking the container closure from a supply chute.

15. The device according to claim 12, wherein more than one container closure is transportable by each carriage.

16. The device accordingly to claim 12, wherein the transport device includes:
a track switch; and
a buffer for interim storage of the container closure, wherein the buffer is connected to a main transport line of the transport device via the track switch.

17. The device according to claim 12, further comprising a cleaning device configured to clean the container closure, the cleaning device disposed upstream of the treatment chamber,
wherein the transport device includes, relative to its main direction of travel, a twisted section such that the container closure that is transported by the transport device undergoes a change in its orientation in the twisted section and the twisted section is disposed in a region of the cleaning device.

18. The device according to claim 11, wherein the transport device includes a buffer for interim storage of the container closure.

19. The device according to claim 18, wherein the transport device includes a track switch and the buffer is connected to a main transport line of the transport device via the track switch.

20. The device accordingly to claim 19, wherein the track switch is a first track switch configured to connect the buffer to the main transport line at a first position in the main transport line, and the transport device includes a second track switch configured to connect the buffer to the main transport line at a second position in the main transport line.

21. The device according to claim 20, wherein the transport device includes, relative to its main direction of travel, a twisted section such that the container closure that is transported by the transport device undergoes a change in its orientation in the twisted section.

22. The device according to claim 21, further comprising a cleaning device configured to clean the container closure, the cleaning device disposed upstream of the treatment chamber and the twisted section is disposed in a region of the cleaning device.

23. The device according to claim 22, wherein the transport device is configured to supply the container closure directly to a capper for closing a container with the container closure.

24. The device according to claim 11, wherein the transport device is configured to supply the container closure directly to a capper for closing a container with the container closure.

25. The device according to claim 11, wherein the treatment chamber is configured as a sterilization chamber for a container.

26. The device according to claim 25, wherein the treatment chamber is further configured as a sterilization chamber for a preform of the container.

27. The device accordingly to claim 25, wherein the treatment chamber is further configured as a sterilization chamber for the container closure, the container closure configured to close the container.

28. The device according to claim 11, further comprising a cleaning device configured to clean the container closure, the cleaning device disposed upstream of the treatment chamber.

29. The device according to claim 11, wherein the transport device includes, relative to its main direction of travel, a twisted section such that the container closure that is transported by the transport device undergoes a change in its orientation in the twisted section.

30. The device according to claim 29, further comprising a cleaning device configured to clean the container closure, the cleaning device disposed upstream of the treatment chamber and the twisted section is disposed in a region of the cleaning device.
